(19) United States
(12) Patent Application Publication
(10) Pub. No.: US 2025/0161377 A1
(43) Pub. Date: May 22, 2025

(54) COMPOSITION COMPRISING AT LEAST ONE BIFIDOBACTERIUM STRAIN FOR THE TREATMENT OF VITILIGO

(71) Applicants: INSTITUT NATIONAL DE LA SANTE ET DE LA RECHERCHE MEDICALE, PARIS (FR); UNIVERSITÉ CÔTE D'AZUR, NICE (FR); ISISPHARMA FRANCE, WASQUEHAL (FR)

(72) Inventors: Thierry PASSERON, Nice Cedex 04 (FR); Meri TULIC, Nice Cedex 03 (FR); Florence NADAL, SAUBENS (FR)

(21) Appl. No.: 18/872,694

(22) PCT Filed: Jun. 5, 2023
(86) PCT No.: PCT/EP2023/064969

§ 371 Date: Dec. 6, 2024

(30) Foreign Application Priority Data
Jun. 9, 2022 (FR) 2205567

(51) Int. Cl. A61K 35/745; A61K 31/216; A61K 36/31; A61P 17/18

(52) U.S. Cl. CPC A61K 35/745; A61K 31/216; A61K 36/31; A61P 17/18

(57) ABSTRACT

The invention relates to a composition aimed at preventing and/or treating early-stage vitiligo in a subject, corresponding to the deregulation of keratinocytes and melanocytes mitochondrial function associated with an abnormally high ROS level within these mitochondria, which composition comprises at least one strain of Bifidobacterium longum or Bifidobacterium breve, or a lysate thereof.

This patent application claims priority of the French patent application FR 2205567 filed on Jun. 9, 2022 and incorporated herein by reference.

### FIELD OF THE INVENTION

The present invention relates to compositions and their use in the treatment or prevention of vitiligo.

### BACKGROUND ART

Vitiligo is a skin condition characterized by the appearance of white spots on the hands, feet, face or any other part of the body. These spots are caused by depigmentation, which corresponds to the disappearance of melanocytes. This depigmentation can be more or less important and the white spots of variable sizes. In some cases, the hair or hair growing inside the depigmented areas may also be white.

Vitiligo is neither contagious nor painful, but it can cause significant psychological distress. Note that people with dark skin suffer particularly because this condition is even more visible in them. However, the impact on quality of life is significant for all affected individuals. Vitiligo is still insufficiently treated by doctors.

Vitiligo affects approximately 0.5 to 2% of the world's population, regardless of ethnicity or gender. It generally appears around the age of 10 to 30, half of those affected are affected before the age of 20. Vitiligo is therefore quite rare in young children. It affects both men and women equally.

There are several types of vitiligo:

1) Segmental vitiligo, localized on only one side of the body, for example on part of the face, upper body, leg, or arm. This form of vitiligo appears more often in children or adolescents. This form appears rapidly over a few months, then generally stops evolving.

2) Non-segmental vitiligo, which presents as spots that are often more or less symmetrical, affecting both sides of the body. The evolution is unpredictable, the spots can remain small and localized or spread rapidly.

Usually, the disease progresses at an unpredictable rate and may stop or spread continuously or most often in flare-ups. Vitiligo can thus evolve in phases, the aggravations sometimes occurring after a psychological or physical triggering event. In very rare cases, the plaques disappear on their own. These people are also more likely to suffer from other autoimmune diseases, including autoimmune thyroid dysfunction.

Vitiligo is a polygenic multifactorial disease causing the loss of melanocytes.

Several hypotheses are currently advanced to explain the loss of melanocytes. Vitiligo is probably a pathology that has both genetic, environmental, and autoimmune origins. Vitiligo is a disease with a strong autoimmune component. The involvement of cellular immunity, particularly the IFN gamma pathway, is now well demonstrated. Innate immunity is also involved and links external or internal stress factors to the activation of adaptive immunity.

According to several studies, the keratinocytes and melanocytes of individuals with vitiligo would primarily accumulate numerous free radicals, which then leads to the production of cytokines and chemokines by keratinocytes and melanocytes and the activation of the immune system.

To date, treatments for vitiligo often require many months and are essentially effective for facial lesions. Failures remain numerous, especially for bony prominences and hands and feet. The standard treatment combines phototherapy (at best UV-B) with local treatments using topical corticosteroids or calcineurin inhibitors. Very active forms may benefit from mini pulses of cortisone. Finally, localized and stable forms of vitiligo and segmental vitiligo are good indications for surgical treatment, particularly by grafting of epidermal cell suspensions.

There is thus a strong demand for a more effective therapy that is well-tolerated and more accessible in terms of cost. The topical route is an alternative, but only small molecules are suitable for penetrating the epidermis and effectively targeting keratinocytes and melanocytes.

### DETAILED DESCRIPTION OF THE INVENTION

The inventors have now demonstrated that extracts from bacterial strains belonging to the species Bifidobacterium longum or Bifidobacterium breve exhibit a particularly significant activity in reducing mitochondrial oxidative stress in a cellular model associated with the early stage of vitiligo, corresponding to the deregulation of mitochondrial function in keratinocytes.

Therefore, a first aspect of the invention relates to a composition for preventing and/or treating, in a subject, the early stage of vitiligo corresponding to the deregulation of keratinocytes and melanocytes mitochondrial function associated with an abnormally high ROS level within these mitochondria, which composition comprises at least one strain of Bifidobacterium longum or Bifidobacterium breve, or a lysate thereof.

It is described in patent application FR 2999933 A1, the increase in melanin production in primary human melanocytes (NHEM) following the application of an extract of Bifidobacterium longum. Such a property of increasing melanin production in melanocytes indeed allows for the possibility of evening out skin tone, it does not suggest preventing or treating vitiligo, particularly not the early stage of the condition. Now, such a property does not at all allow for the possibility of preventing or treating vitiligo and even less the early stage of vitiligo. Indeed, this activity has no effect on the disappearance of melanocytes that characterizes this pathology. On the contrary, and in light of this teaching, a person skilled in the art would reasonably avoid using such an extract of Bifidobacterium longum in patients suffering from vitiligo, as it could further weaken an already fragile cell population (i.e., melanocytes) and accelerating, rather than slowing, the onset of the pathology.

Preferably, the said composition is aimed at preventing and/or treating a subject suffering from vitiligo.

In relation to vitiligo, it can correspond just as well to segmental vitiligo or to non-segmental vitiligo.

As for the subject, it concerns a mammal and, preferably, a human.

Bifidobacterium is a genus of Gram-positive anaerobic actinobacteria from the bifidobacterial family. These bacteria are composed of irregularly shaped bacilli, strict anaerobes, exhibiting a negative catalase test, non-motile. The Bifidobacteria are part of the lactic bacteria family. They participate in the fermentation of milk in the production of cheeses and preparations similar to yogurts. Furthermore, they produce large amounts of lactic acid, resulting in a decrease in pH that is favorable to them and inhibits the growth of other germs. The species Bifidobacterium longum or Bifidobacterium breve are present in the human digestive tract.

Advantageously, the content of the composition according to the invention of at least one strain of Bifidobacterium longum or Bifidobacterium breve is comprised between 10² and 10¹³ CFU/ml of composition, preferably it is comprised between 10⁵ and 10¹¹ CFU/ml of composition.

Furthermore, the at least one strain of Bifidobacterium longum or Bifidobacterium breve may not be maintained viable after its integration into the composition. In this context, the composition can be stored at room temperature.

By “lysate,” it is meant a culture of such a bacterial strain that has undergone cellular lysis.

A cellular lysis can be achieved by various technologies such as osmotic shock, thermal shock, ultrasonic treatment, or mechanical stress (e.g., centrifugation).

Advantageously, such a lysate is obtained by thermal shock or ultrasonic lysis.

Typically, such a lysate can be produced using the technology described in U.S. Pat. No. 4,464,362, specifically following the subsequent protocol.

The microorganisms belonging to the Bifidobacterium species are cultured under anaerobic conditions in a suitable culture medium (refer, for example, to U.S. Pat. No. 4,464,362 and EP 0 043 128 B1). When the stationary phase of development is reached, the cultures are inactivated by pasteurization, for example at a temperature of 60 to 65° C. for 30 minutes. The microorganisms are then collected by a conventional separation technique, for example by membrane filtration, centrifugation, and resuspension in a sterile physiological saline solution. The lysate is then obtained by ultrasonic disintegration of the microorganisms in order to release the cytoplasmic fractions, cell wall fragments, and metabolic products. All these components are then stabilized in a mildly acidic solution.

Preferably, it is a lysate of Bifidobacterium longum (N^{o} CAS 96507-89-0). As an example of such a lysate, we can cite the product registered under the name “REPAIR COMPLEX CLR™” by K. RICHTER GmbH, the product registered under the name PROMACARE® BFL by UNIPROMA or the product registered under the name BIFIDAFERM™ by THE GARDEN OF NATURALSOLUTIONS.

According to another preference, it will be a lysate of Bifidobacterium breve.

Advantageously, the content of the composition according to the invention in lysate of at least one strain of Bifidobacterium is comprised between 1 and 20% (by weight relative to the total weight of the composition), preferably it is comprised between 5 and 10%.

By “Antioxidant,” it is meant a molecule capable of blocking or slowing the oxidation of another molecule upon contact.

This class of molecules includes a very wide range of components, among which are vitamins, metallic species, peptides or peptide derivatives, polyphenols, flavonols, or organosulfur compounds.

As examples of vitamins that exhibit antioxidant activity, one can cite vitamin A, vitamin C, vitamin E, coenzyme Q10, and their derivatives.

As examples of metallic species that exhibit antioxidant activity, selenium and zinc can be mentioned.

As examples of pigments exhibiting antioxidant activity, cite β-carotene, lycopene, or curcumin can be mentioned.

As examples of peptides or peptide derivatives exhibiting antioxidant activity, L-carnosine, Glutathione, or Superoxide dismutase (SOD) can be mentioned. L-carnosine is a dipeptide of beta-alanine and histidine, available under the name SPECPED® LCS (SPEC-CHEM IND) or DRAGOSINE® (SIMRISE). Glutathione is a pseudo-tripeptide formed by the condensation of glutamic acid, cysteine, and glycine, available under the name SPECKAREE® GSH (SPEC-CHEM IND) or AC-GSH (SOHO ANECO CHEMICALS) are metalloproteins that catalyze the dismutation of superoxide anions into oxygen and hydrogen peroxide. SODs are available particularly under the names BIOCELL SOD (LONZA) or DISMUTIN® PF (DSM).

As examples of polyphenols exhibiting antioxidant activity, one can cite anthocyanins in red fruits, proanthocyanidins in chocolate, caffeoylquinic and feruloylquinic acids in coffee, flavonoids in citrus fruit, catechins in green tea, and quercetin in apples.

As examples of flavonols exhibiting antioxidant activity, one can mention quercetol or PYCNOGENOL®.

As examples of organosulfur compounds exhibiting antioxidant activity, one can mention alpha-lipoic acid, methionine, or cysteine.

Advantageously, the content of the composition according to the invention in antioxidants is between 0.05 and 10% (by weight relative to the total weight of the composition). Indeed, the content of antioxidant to be adopted can vary greatly depending on the antioxidant capacity of the chosen molecule.

Now, the inventors have demonstrated that sinapine and its derivatives also exhibit a particularly significant activity in reducing mitochondrial oxidative stress in the same cellular model. Therefore, a combination of this antioxidant with the bacterial species (or their lysates) previously mentioned presents a definite interest for the treatment of this early stage of vitiligo.

According to a preferred embodiment, the antioxidant is sinapine, a salt thereof, or one of its derivatives.

Sinapine is an alkaloid with the formula (I)

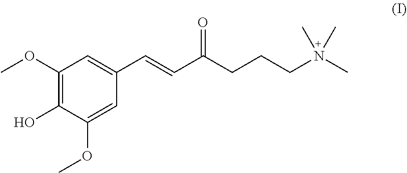

By a derivative of sinapine, it is meant a compound having the formula (II)

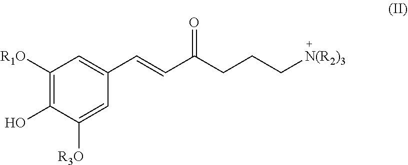

In which
R1 and R3 are independently selected from a hydrogen atom or an alkyl group in C1 to C8, preferably an alkyl group in C1 to C4, and more preferably an alkyl group in C1 to C2; and
R2 is independently chosen from a hydrogen atom or an alkyl group in C1 to C8, preferably an alkyl group in C1 to C4, and more preferably an alkyl group in C1 to C2.

Sinapine is present in significant amounts in the seeds or sprouts of plants from the Brassicaceae family, such as rapeseed (Brassica napus), garden cress (Lepidium sativum), and mustard (Brassica alba).

Advantageously, the sinapine is in the form of an extract from a seed or sprout of a plant belonging to the Brassicaceae family.

The term “extract” refers to a substance extracted from a natural product, regardless of the extraction method or the composition of the ingredients. For example, this includes those obtained by extracting soluble ingredients from a natural product using water or an organic solvent, or those obtained by extracting specific ingredients only, such as oil, from a natural product. The extraction processes are well known to those skilled in the art and typically involve macerating the seeds, whether crushed or not, in the extraction solvent.

Preferably, the extract from the seeds or sprouts of a plant belonging to the Brassicaceae family is an aqueous, alcoholic, or hydro-alcoholic extract. Advantageously, an extract using glycerin as the extraction solvent, such as glycerinated, hydroglycerinated, or hydroalcoholic glycerinated extracts, will be used.

As examples of such extracts, one can mention an aqueous extract of mustard sprouts like the one marketed by MIBELLE under the name LIPERFECTION or the hydroglycerinated extract of garden cress sprouts marketed by MIBELLE under the name DETOXOPHANE.

According to a preferred embodiment, the composition according to the invention is intended for topical application on the skin, particularly the skin of the face, hands, neck, and body in general. Accordingly, the composition according to the invention takes the form of a cream, serum, milk, lotion, or gel. Preferably, the composition according to the invention will take the form of a cream.

The composition according to the invention is then applied to the affected skin surface at least once a day, preferably at least twice a day (e.g., once in the morning and once in the evening).

Generally, the topical composition according to the invention may include many types of adjuvants or active ingredients used in pharmaceutical or cosmetic formulations, preferably dermatological, whether they are fatty substances, organic solvents, thickeners, gelling agents, softeners, opacifiers, stabilizers, foaming and/or detergent surfactants, emollients, superfatting agents, perfumes, ionic or non-ionic emulsifiers, fillers, sequestering agents, chelators, preservatives, essential oils, coloring materials, pigments, hydrophilic or lipophilic actives, humectants such as glycerin or glycols, preservatives, dyes, cosmetic actives, mineral and/or organic sunscreen filters, mineral fillers, synthetic fillers, silicone elastomers, or plant extracts or lipid vesicles, or any other ingredient commonly used in cosmetics.

Now and advantageously, the composition according to the invention will not contain parabens.

Always advantageously, the composition according to the invention will not contain phenoxyethanol.

As examples of oils, one can mention paraffins, isoparaffins, mineral white oils, vegetable oils (from flowers, fruits, vegetables, trees, grains, oilseeds . . . ), animal oils, synthetic oils, silicone oils, and fluorinated oils; and more particularly: vegetable-based oils, such as sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy oil, pumpkin oil, evening primrose oil, millet oil, barley oil, rye oil, safflower oil, bancoulier oil, passionflower oil, hazelnut oil, palm oil, shea butter, apricot kernel oil, calophyllum oil, Sisymbrium officinale oil, avocado oil, calendula oil, oils from flowers or vegetables; ethoxylated vegetable oils; animal-derived oils, such as squalene, squalane; mineral oils, such as paraffin oil, petrolatum oil, and isoparaffins; synthetic oils, notably fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate; lanolin acid derivatives, such as isopropyl lanolate, isocetyl lanolate; monoglycerides, diglycerides, and triglycerides of fatty acids like glyceryl triheptanoate; alkylbenzoates; polyalphaolefins; polyolefins like polyisobutene; synthetic isoalkanes like isohexadecane, isododecane; perfluorinated oils and silicone oils. Among the latter, one can particularly mention dimethylpolysiloxanes, methylphenylpolysiloxanes, aminomodified silicones, fatty acid modified silicones, alcohol modified silicones, alcohols and fatty acids modified silicones, polyether group modified silicones, epoxy modified silicones, fluorine group modified silicones, cyclic silicones, and alkyl group modified silicones.

As another fatty substance, one can mention fatty alcohols, whether linear or branched, saturated or unsaturated, mixtures of fatty alcohols, linear and/or branched, saturated and/or unsaturated, or fatty acids, linear or branched, saturated or unsaturated, mixtures of fatty acids linear or branched, saturated or unsaturated.

Among the thickening and/or emulsifying polymers that can be used, there are, for example, homopolymers or copolymers of acrylic acid or derivatives of acrylic acid, homopolymers or copolymers of methacrylic acid or derivatives of methacrylic acid, homopolymers or copolymers of acrylamide, homopolymers or copolymers of acrylamide derivatives, homopolymers or copolymers of acrylamidomethyl propanesulfonic acid, homopolymers or copolymers of vinylic monomers, homopolymers or copolymers of trimethylaminoethylacrylate chloride, vegetable or biosynthetic origin hydrocolloids such as xanthan gum, karaya gum, carrageenans, alginates; silicates; cellulose and its derivatives; starch and its hydrophilic derivatives; polyurethanes.

Among the types of polyelectrolyte polymers that can be used in the production of a gelled aqueous phase suitable for use in the preparation of W/O, O/W, W/O/W, or O/W/O emulsions, or an aqueous gel including SEPIBIO™ POTENTILLA 217, are, for example, copolymers of acrylic acid and 2-methyl-[(1-oxo-2-propenyl)amino]-1-propane sulfonic acid (AMPS), copolymers of acrylamide and 2-methyl-[(1-oxo-2-propenyl)amino]-1-propane sulfonic acid, copolymers of 2-methyl-[(1-oxo-2-propenyl)amino]-1-propane sulfonic acid and hydroxyethyl acrylate, homopolymer of 2-methyl-[(1-oxo-2-propenyl)amino]-1-propane sulfonic acid, homopolymer of acrylic acid, copolymers of ethyl trimethyl ammonium acryloyl chloride and acrylamide, copolymers of AMPS and vinylpyrrolidone, copolymers of AMPS and N,N-dimethylacrylamide, terpolymers of AMPS, acrylic acid, and N,N-dimethylacrylamide, copolymers of acrylic acid and alkyl acrylates with carbon chains containing between ten and thirty carbon atoms, copolymers of AMPS and alkyl acrylates with carbon chains containing between ten and thirty carbon atoms.

Among the waxes usable in the compositions according to the invention, one can mention, for example, beeswax, carnauba wax, Candelilla wax, Ouricouri wax, Japan wax, China wax, rice bran wax, Montan wax, fiber waxes from cork or sugarcane, paraffin waxes, lignite waxes, microcrystalline waxes, lanolin wax, ozokerite, polyethylene wax, hydrogenated oils, silicone waxes, alkenone waxes, vegetable waxes, fatty alcohols, and solid fatty acids at room temperature, solid glycerides at room temperature.

Among the emulsifiers usable in the compositions according to the invention, one can mention:
fatty esters of alkylpolyglycosides, possibly alkoxylated;
alkoxylated fatty esters;
carbamates of polyalkylene glycols with fatty chains;
fatty acids, ethoxylated fatty acids, fatty acid and sorbitol esters, ethoxylated fatty acid esters, polysorbates, polyglycerol esters, ethoxylated fatty alcohols, sucrose esters, alkylpolyglycosides, sulfated and phosphated fatty alcohols or mixtures of alkylpolyglycosides and fatty alcohols;
combinations of emulsifying surfactants chosen among alkylpolyglycosides;
combinations of alkylpolyglycosides and fatty alcohols, esters of polyglycerols, polyglycols, or polyols.

Among the surfactants usable in the compositions according to the invention, one can mention: topically acceptable anionic, cationic, amphoteric, or nonionic surfactants commonly used in this field of activity.

Among the anionic surfactants usable in the compositions according to the invention, particularly mentioned are the salts of alkaline metals, alkaline earth metals, ammonium salts, amino acid salts, amino alcohol salts of the following compounds: alkyl ether sulfates, alkyl sulfates, alkylamido ether sulfates, alkylaryl polyether sulfates, monoglyceride sulfates, alpha-olefin sulfonates, paraffin sulfonates, alkyl phosphates, alkyl ether phosphates, alkyl sulfonates, alkylamide sulfonates, alkylaryl sulfonates, alkyl carboxylates, alkyl sulfosuccinates, alkyl ether sulfosuccinates, alkylamide sulfosuccinates, alkyl sulfoacetates, alkyl sarcosinates, acyl isethionates, N-acyl taurates, acyl lactylates.

Among the amphoteric surfactants usable in the compositions according to the invention, one can mention alkyl betaines, alkylamido betaines, sultaines, alkylamidoalkyl sulfobetaines, imidazoline derivatives, phosphobetaines, amphoacetates, and amphopropionates.

According to another preferred embodiment, the composition according to the invention is intended for oral administration which may take the form of a nutritional supplement or a dietary preparation.

As an example, the said nutritional supplement or dietary preparation can be in the form of granules, tablets, soft capsules, capsules, suspensions, syrups, ampoules, mouth sprays, drops, chewable gums, powders to dissolve, or solutions.

In the case of a powder to dissolve in a liquid, which liquid can then be, for example, water, milk, or a milk-based preparation, or even fruit juice.

Other pharmaceutically and/or food-grade acceptable agents can be added, such as bulking agents, flow aids, natural extracts, minerals, trace elements, fatty acids, anti-caking agents, natural oils, flavors, colorants, acidifiers, thickeners, preservatives, and sweeteners.

As examples of bulking agents, one can mention microcrystalline cellulose, potato maltodextrin, or magnesium lactate.

As examples of flow aids, one can mention magnesium silicate, magnesium stearate, or colloidal silica.

As examples of minerals or trace elements, one can mention magnesium, iodine, iron, copper, zinc, selenium, chromium, molybdenum, manganese, silicon, vanadium, nickel, or tin.

As examples of amino acids, one can mention alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine.

As examples of fatty acids, one can mention unsaturated fatty acids such as omega-3 or omega-6.

As examples of anti-caking agents commonly used in the food industry, one can mention magnesium stearate (E470b), silicon dioxide (E551), and colloidal silica.

As an example of a thickener, one can mention potato starch, hydroxypropylmethylcellulose, citrus pectin, guar gum, locust bean gum, xanthan gum, agar-agar, konjac, hydrogenated oils, or beeswax.

As an example of acidifiers, one can mention citric acid.

As examples of sweeteners, one can mention, among others, xylitol, aspartame, glucose syrup, fructooligosaccharide syrup, powdered or syrup maltitol, acesulfame potassium, fructooligosaccharide, sodium cyclamate, and sucralose.

As examples of colorants, one can cite curcumins (E100), carminic acid (E120), erythrosine (E127), chlorophylls and chlorophyllins (E140), copper complexes of chlorophylls and chlorophyllins (E141), caramel (E150), carotenoids (E160), anthocyanins (E163), calcium carbonate (E170), iron oxide and hydroxide (E172), and orcein (E182).

As examples of preservatives, one can mention potassium sorbate, sodium benzoate, or ascorbyl palmitate (antioxidant).

In order to further enhance the activity obtained by the composition according to the invention, it may be combined with other active ingredients, especially those known for their anti-aging, firming, restructuring, stimulating, energizing, anti-wrinkle, relaxing, moisturizing, antimicrobial, sebum-regulating, purifying, soothing, relaxing, anti-stress, brightening, immunomodulatory, cell renewal stimulating, lifting, plumping, and complexion enhancing properties.

According to a second aspect, the composition described previously is used in a method aimed at preventing and/or treating vitiligo in a subject and includes a step of administering a therapeutically effective amount of the composition onto a skin surface of the said subject.

By “therapeutically effective amount,” it is meant a sufficient quantity to achieve the desired biological effect.

The invention will be better understood in light of the following examples, which are given purely for illustrative purposes and are not intended to limit the scope of the invention, as defined by the appended claims.

### EXAMPLES

1) Capacity to Induce a Drastic Reduction in ROS within the Mitochondria of Keratinocytes from Certain Specific Bacterial Extracts

Primary human keratinocytes (NHK) were isolated from the foreskin of healthy children and cultured in a serum-free medium supplemented with bovine pituitary extract (250 μg/mL) and EGF (0.25 ng/mL).

The NHKs are then seeded onto 6-well plates and incubated in a controlled atmosphere. When the keratinocytes reach 70 to 80% confluence, the cells are pretreated, or not, for one hour with various components. As a positive control, the NHKs are pretreated for one hour with sinapine (100 μM).

The plates are then washed 3 times with a PBS solution before incubating the pretreated NHKs for one hour in the presence of H₂O₂ (100 μM) or LPS (100 ng/mL).

At the end of the incubation, the cells are incubated in a solution of dihydrorhodamine (5 μM) for 30 minutes at 37° C., which allows for the labeling of ROS, particularly mitochondrial ROS.

After 3 washes with a PBS solution, the mitochondrial ROS are quantified by FACS.

The results are presented in the following Table 1.

TABLE 1

| Pre-treatment | Treatment | Mitochondrial ROS Level (%) |
|---|---|---|
| — | basal | 100 |
| | H₂O₂ | 352 |
| | LPS | 124 |
| Sinapine | — | 100 |
| | H₂O₂ | 100 |
| | LPS | 97 |
| Extract of B. longum | — | 32 |
| | H₂O₂ | 12.7 |
| | LPS | 15.8 |
| Extract of B. breve | — | 35 |
| | H₂O₂ | 7 |
| | LPS | 5.2 |
| Sinapine + longum . . . | H₂O₂ | 2 |
| Sinapine + breve | H₂O₂ | 1 |

In a completely unexpected manner, the results showed that extracts from Bifidobacterium longum and Bifidobacterium breve induced a drastic reduction in ROS within the mitochondria of keratinocytes subjected to stress or not, and even to major oxidative stress (H₂O₂). It is noteworthy that the observed reduction is specific to the bacterial species used. Indeed, this reduction is not observed with bacterial extracts from the species Bifidobacterium bifidum and Bifidobacterium lactis. The observed reduction in ROS within the mitochondria of keratinocytes with extracts from Bifidobacterium longum and Bifidobacterium breve is far superior to that observed with sinapine.

It should be noted that additional experiments conducted with plant extracts containing sinapine (LIPERFECTION and DETOXOPHANE) have shown similar activity of these extracts to that observed with sinapine alone.

The results finally demonstrate that the combination of Bifidobacterium longum and Bifidobacterium breve extracts with sinapine leads to a composition with an even greater potential for reducing ROS within the mitochondria of keratinocytes.

The same tests are carried out on melanocytes.

2) Capacity to Induce a Drastic Reduction in the Expression of Pro-Inflammatory Cytokines within Keratinocytes by Specific Bacterial Extracts

Primary human keratinocytes (NHK) were isolated from the foreskin of healthy children and cultured in a serum-free medium supplemented with bovine pituitary extract (250 μg/mL) and EGF (0.25 ng/mL).

The NHKs are then seeded onto 6-well plates and incubated in a controlled atmosphere. When the keratinocytes reach 70 to 80% confluence, the NHKs are incubated for one hour in the presence of LPS (100 ng/mL) or IFN/TNF (10 ng/ml) for 24 hours.

At the end of the incubation period, the cells are incubated or not for 24 hours with 100 μg/mL of extracts from different strains of Bifidobacterium. After this incubation period, the supernatants are collected for ELISA assays (TNF alpha, IFN gamma, IL1 beta, CXCL10, CXCL11, and CXCL16).

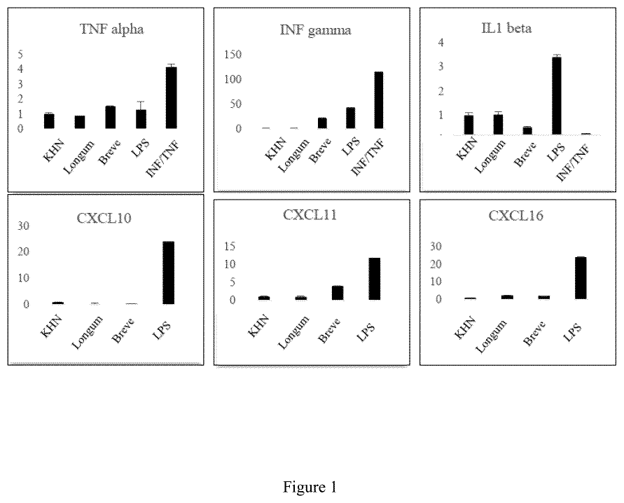

The results are presented in FIG. 1 which describes the increase in the expression of each of these pro-inflammatory cytokines (TNF alpha, IFN gamma, IL1 beta, CXCL10, CXCL11, and CXCL16) in these different conditions compared to untreated cells (KHN; reference level 1.0).

As previously, the results showed that extracts from Bifidobacterium longum and Bifidobacterium breve induced, as with mitochondrial ROS, a drastic reduction in the expression of various pro-inflammatory cytokines within keratinocytes subjected or not to stress. Again, the reduction observed is specific to the bacterial species used. Indeed, this significant reduction is not observed with bacterial extracts from the species Bifidobacterium bifidum and Bifidobacterium lactis.

Again, the same tests are performed on melanocytes.

### 3) Composition According to the Invention

Based on the previous results, a composition was developed that includes an extract of Bifidobacterium longum and a plant extract containing sinapine designed to prevent the appearance of new lesions or to stabilize them in subjects at an early stage of vitiligo, namely those presenting melanocytes with impaired mitochondrial function. The details of a composition according to the invention are given in the following Table 2.

TABLE 2

| | Presence in the composition (% by |
|---|---|
| Compounds | weight relative to the total weight) |
| REPAIR COMPLEX CLR ™ | 5 à 10 |
| LIPERFECTION | 2 à 5 |
| Glycerin | 4 à 5 |
| C12-C15 alkyl benzoate | 1 à 4 |
| Ethylhexyl palmitate | 4 à 5 |
| Glyceryl stearate | 1 à 3 |
| Cyclomethicone | 5 à 8 |
| Potassium cetyl phosphate | 1 à 3 |
| Hydroxyethyl acrylate | 0.8 à 2.3 |
| Xanthan gum | 0.1 à 0.35 |
| Magnesium silicate | 0.3 |
| α-Tocopheryl acetate | 0.2 à 0.4 |
| Water | Qsp 100 |

The daily application of the composition according to the invention on the skin, especially on areas of skin containing melanocytes with impaired mitochondrial function, helps to delay the appearance of new lesions and stabilize existing lesions in subjects at an early stage of vitiligo.

1. A composition for use in preventing and/or treating early-stage vitiligo in a subject corresponding to the deregulation of keratinocytes and melanocytes mitochondrial function associated with an abnormally high ROS level within these mitochondria, which composition comprises at least one strain of Bifidobacterium longum or of Bifidobacterium breve, or a lysate thereof.

2. The composition according to claim 1, characterized in that said composition aims at the prevention or treatment of a patient suffering from vitiligo.

3. The composition according to claim 1, characterized in that said at least one strain of Bifidobacterium longum or Bifidobacterium breve is present in the form of a lysate, preferably said lysate is obtained by ultrasonic lysis.

4. The composition according to claim 1, characterized in that the content of the composition according to the invention in lysate of said at least one strain of Bifidobacterium is between 1 and 20% (by weight relative to the total weight of the composition), preferably between 5 and 10%.

5. The composition according to claim 1, characterized in that it further comprises at least one antioxidant.

6. The composition according to claim 5, characterized in that said antioxidant is chosen from sinapine and its derivatives.

7. The composition according to claim 6, characterized in that the sinapine derivatives have the formula (II): 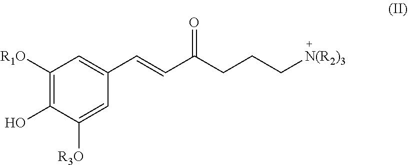
Wherein
R1 and R3 are independently chosen from a hydrogen atom or an alkyl group in C1 to C8, preferably an alkyl group in C1 to C4, and more preferably an alkyl group in C1 to C2; and
R2 is independently chosen from a hydrogen atom or an alkyl group in C1 to C8, preferably an alkyl group in C1 to C4, and more preferably an alkyl group in C1 to C2.

8. The composition according to claim 6, characterized in that the sinapine is in the form of an extract from seeds or sprouts of a plant belonging to the Brassicaceae family.

9. The composition according to claim 1, characterized in that it is intended for topical administration.

10. The composition according to claim 1, characterized in that it is intended for oral administration.
